(19) United States
(12) Patent Application Publication
(10) Pub. No.: US 2012/0165831 A1
(43) Pub. Date: Jun. 28, 2012

(54) EXFOLIATING PILLOW

(76) Inventor: Eric Gonzalez, Laredo, TX (US)

(21) Appl. No.: 13/329,528

(22) Filed: Dec. 19, 2011

Related U.S. Application Data
(60) Provisional application No. 61/426,889, filed on Dec. 23, 2010.

(51) Int. Cl. A61B 17/50

(52) U.S. Cl. 606/131

(57) ABSTRACT

A method for treating an affected skin area includes providing a pillow including an exfoliating micro-fiber material coupled to the pillow. The affected skin area is positioned in contact with the exfoliating micro-fiber material. The affected skin area is then abraded by moving the affected skin area relative to the exfoliating micro-fiber material. By providing the exfoliating micro-fiber material coupled to the pillow, the abrading of the affected skin area may be performed during the sleep of a person having the affected skin area.

### CROSS REFERENCE TO RELATED APPLICATION

This patent application claims priority to U.S. Provisional Patent Appl. Ser. No. 61/426,889, filed on Dec. 23, 2010, attorney docket number 46741.03, the disclosure of which is incorporated herein by reference in its entirety.

### BACKGROUND

The present disclosure relates generally to treatment of skin disorders, and more particularly to an exfoliating pillow for the treatment of skin disorders.

Skin disorders such as, for example, acne, pimples, and/or a variety of other skin disorders known in the art, affect many people of all ages and genders. Convention treatments for such skin disorder include applying creams, lotions, and/or other medicines to the affected area of the skin. Some treatments also include reducing external pressure on skin areas affected with the skin disorder such that capillary blood flow is not restricted and capillary pressure does not increase, in order to prevent rupture of the blood capillaries. Typically, this is done by providing numerous point contacts that support the affected area of the skin without interfering with capillary circulation. However, the present disclosure provides an exfoliating pillow that teaches away from such conventional techniques and has been found to provide advantages in the treatment of skin disorders that are not achieved using such conventional treatments.

### SUMMARY

According to one embodiment, an exfoliation system includes a pillow case that defines a pillow housing, an exfoliation section located on the pillow case opposite the housing, and an exfoliating micro-fiber material included in the exfoliation section, wherein the exfoliating micro-fiber material includes a plurality of micro-fibers that are operable, in response to contact with and relative motion of an affected skin area, to abrade the affected skin area.

In another embodiment, a skin treatment system includes a pillow, an exfoliation section coupled to the pillow, and an exfoliating micro-fiber material included in the exfoliation section, wherein the exfoliating micro-fiber material includes a plurality of micro-fibers that are operable, in response to contact with and relative motion of an affected skin area, to abrade the affected skin area.

In another embodiment, a method for treating an affected skin area includes providing a pillow including an exfoliating micro-fiber material coupled to the pillow, positioning the affected skin area in contact with the exfoliating micro-fiber material, and abrading the affected skin area by moving the affected skin area relative to the exfoliating micro-fiber material.

### BRIEF DESCRIPTION OF THE DRAWINGS

FIG. 1 is a perspective view of an embodiment of a pillow case.

FIG. 2a is a perspective view of an embodiment of an exfoliating section of the pillow case of FIG. 1.

FIG. 2b is a perspective view of an embodiment of an exfoliating section of the pillow case of FIG. 1.

FIG. 2c is a perspective view of an embodiment of micro-fiber used in the exfoliating section FIG. 2a or 2b.

FIG. 2d is a perspective view of an embodiment of micro-fiber used in the exfoliating section FIG. 2a or 2b.

FIG. 3a is a flow chart illustrating an embodiment of a method for treating an affected skin area.

FIG. 3b is a perspective view illustrating an embodiment of the pillow case of FIG. 2a including a pillow.

FIG. 3c is a top view illustrating an embodiment of a person having an affected skin area using the pillow case and pillow of FIG. 3b.

FIG. 3d is a partial cross-sectional perspective view illustrating an embodiment of the affected skin area on the person in FIG. 3c.

FIG. 3e is a cross-sectional view of an embodiment of the engagement and relative movement of the affected skin area of FIG. 3d and the pillow case of FIG. 1.

FIG. 3f is a partial cross-sectional perspective view illustrating an embodiment of the affected skin area of FIG. 3d subsequent to being abraded using the pillow case of FIG. 1.

### DETAILED DESCRIPTION

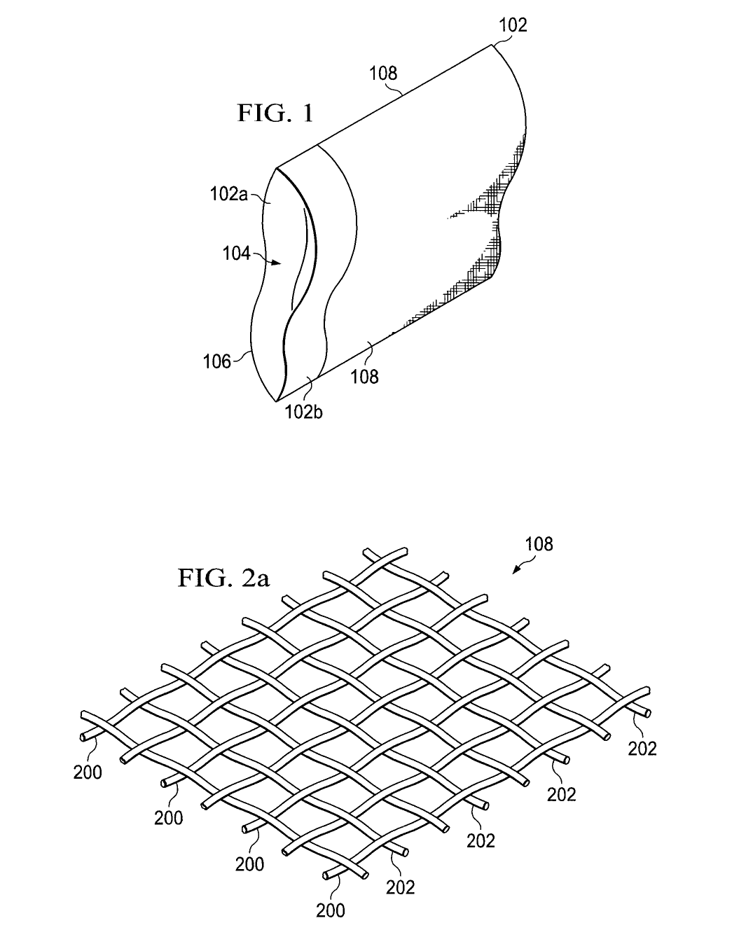

Referring initially to FIG. 1, an embodiment of an exfoliation system 100 is illustrated. The exfoliation system 100 includes a pillow case 102 that defines a pillow housing 104 such that the pillow case 102 includes a inner surface 102a that is located immediately adjacent the pillow housing 104, and an outer surface 102b that is located opposite the pillow case 102 from the inner surface 102a. A pillow entrance 106 is located on an end of the pillow case 102 and immediately adjacent the pillow housing 104. While an embodiment of the pillow entrance 106 has been illustrated in FIG. 1, a variety of pillow entrances are envisioned as falling within the scope of the present disclosure such as, for example, a pillow entrance located at a different position on the pillow case 102 (e.g., the middle of a side of the pillow case 102) or a pillow entrance including a fastener (e.g., a zipper, buttons, and/or a variety of other fasteners known in the art). Furthermore, the pillow case 102 may instead be a pillow by filling the pillow housing 104 with a pillow filling known in the art and then sealing the pillow entrance 106. The pillow case 102 includes an exfoliation section 108 that, in the illustrated embodiment, includes the outer surface 102b of the material that makes up the pillow case 102. However, one of skill in the art will recognize that the exfoliation section may be included on a variety of surfaces on the pillow case 102 such as, for example, the inner surface 102a and outer surface 102b, only one side of the outer surface 102b, only a portion of the outer surface 102b, a material attached to the outer surface 102b, combinations thereof, and/or in a variety of dimensions and/or locations on the pillow case 102. In an embodiment, the exfoliation section 108 includes an exfoliating micro-fiber material. In an embodiment, the exfoliating micro-fiber material includes 70% polyester material and 30% polyamide material. However, other combinations of materials may be suitable such as, for example, 80% polyester material and 80% polyamide material. While the exfoliation section 108 is described as being attached to a pillow or pillow case, it may also be included on bed sheets, blankets, and a variety of bed materials known in the art without departing from the scope of the present disclosure.

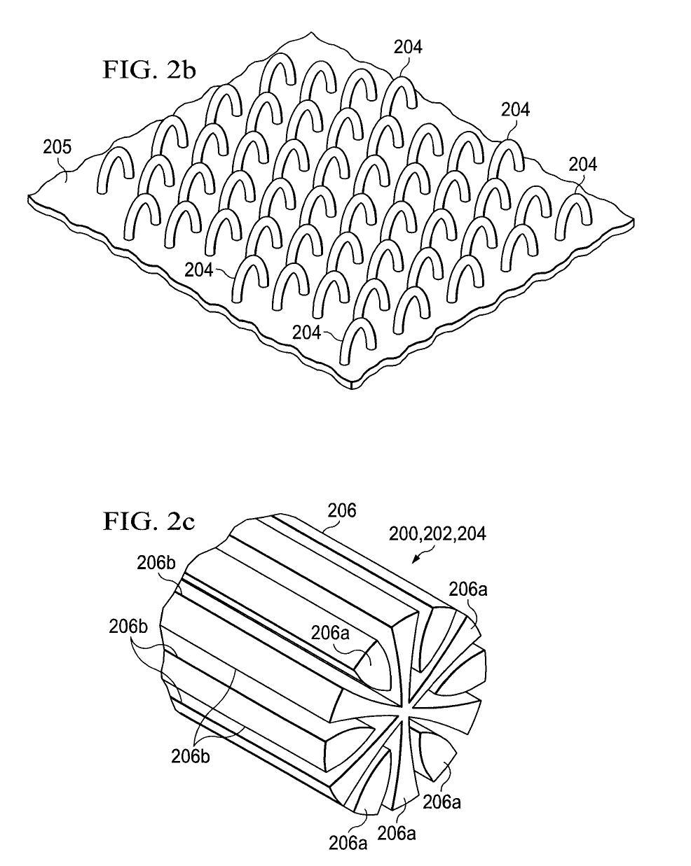

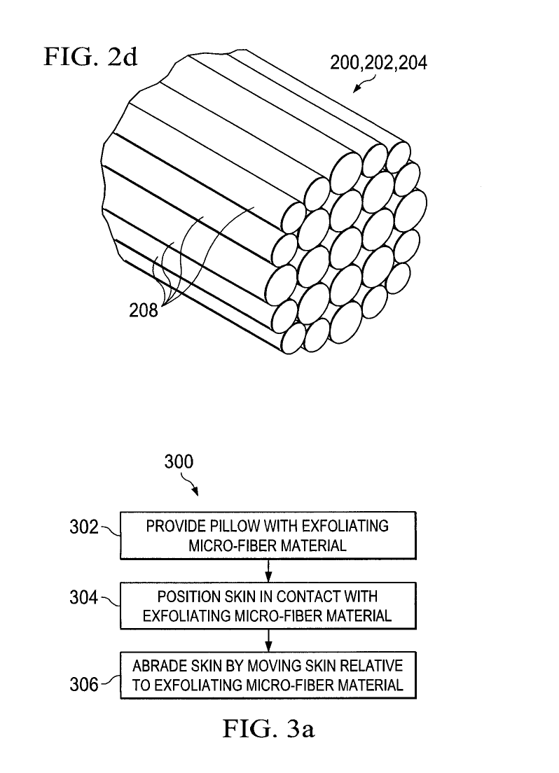

Referring now to FIGS. 1 and 2a, an embodiment of the exfoliation section 108 is illustrated in more detail. As discussed above, the exfoliation section 108 includes an exfoliating micro-fiber material. In the embodiment illustrated in FIG. 2a, the exfoliating micro-fiber material includes a plurality of micro-fibers 200 and 202 in a weaved orientation. In an embodiment, the plurality of micro-fibers 200 and 202 may be fabricated from the same material or materials. In an embodiment, the plurality of micro-fibers 200 and 202 may be fabricated from different materials. In an embodiment, micro-fibers in the plurality of micro-fibers 200 may be fabricated from the same material or materials. In an embodiment, micro-fibers in the plurality of micro-fibers 200 may be fabricated from different materials. In an embodiment, micro-fibers in the plurality of micro-fibers 202 may be fabricated from the same material or materials. In an embodiment, micro-fibers in the plurality of micro-fibers 202 may be fabricated from different materials. In an embodiment, the exfoliating micro-fiber material may include a plurality of layers the same as or similar to the layer of micro-fibers 200 and 202 in the weaved orientation that is illustrated in FIG. 2a. While a particular weaving pattern has been illustrated in FIG. 2a, one of skill in the art will recognize that a variety of different weaving patterns may be used without departing from the scope of the present disclosure.

Referring now to FIGS. 1 and 2b, another embodiment of the exfoliation section 108 is illustrated in more detail. As discussed above, the exfoliation section 108 includes an exfoliating micro-fiber material. In the embodiment illustrated in FIG. 2b, the exfoliating micro-fiber material includes a plurality of micro-fibers 204 in a looped orientation on a base 205. In an embodiment, micro-fibers in the plurality of micro-fibers 204 may be fabricated from the same material or materials. In an embodiment, micro-fibers in the plurality of micro-fibers 204 may be fabricated from different materials. While examples of embodiments of the exfoliation section 108 have been described and illustrated in FIGS. 2a and 2b, one of skill in the art will recognize that variety of other exfoliation sections having different exfoliating micro-fibers than those discussed above may be used without departing from the scope of the present disclosure. Furthermore, the exfoliation section 108 may includes portions having different exfoliation sections (e.g., the exfoliation section may include a portion similar to that illustrated in FIG. 2a, a portion similar to that illustrated in FIG. 2b, and/or other portions known in the art.)

Referring now to FIGS. 2a, 2b, 2c, and 2d, in an embodiment, each of the micro-fibers 200, 202, and/or 204 in the exfoliating micro-fiber material may include a micro-fiber 206 having one or more micro-fiber elements 206a that provide a plurality of edges 206b that make up an outer surface of the micro-fiber 206, as illustrated in FIG. 2c. Furthermore, each of the micro-fibers 200, 202, and/or 204 may include a plurality of ultra-micro-fibers 208 that are bundled together to create the micro-fibers 200, 202, and/or 204, as illustrated in FIG. 2d. In an embodiment, each ultra-micro-fiber 208 may be one of the micro-fibers 206 illustrated in FIG. 2c. While a specific micro-fiber and bundle of ultra-micro-fibers have been described and illustrated, one of skill in the art will recognize that the present disclosure is not so limited and that a variety of micro-fiber structures may be provided that fall within the scope of the present disclosure.

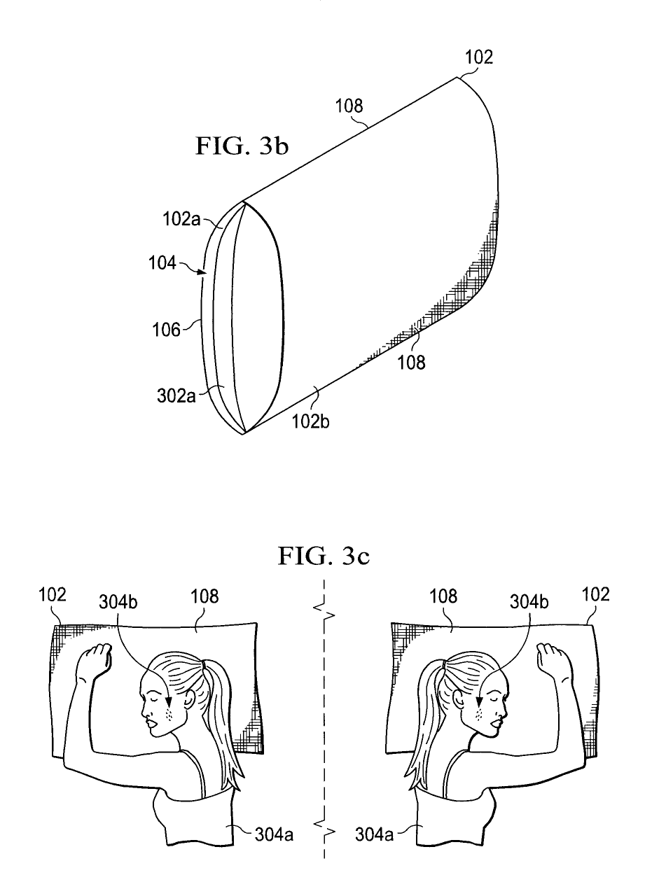

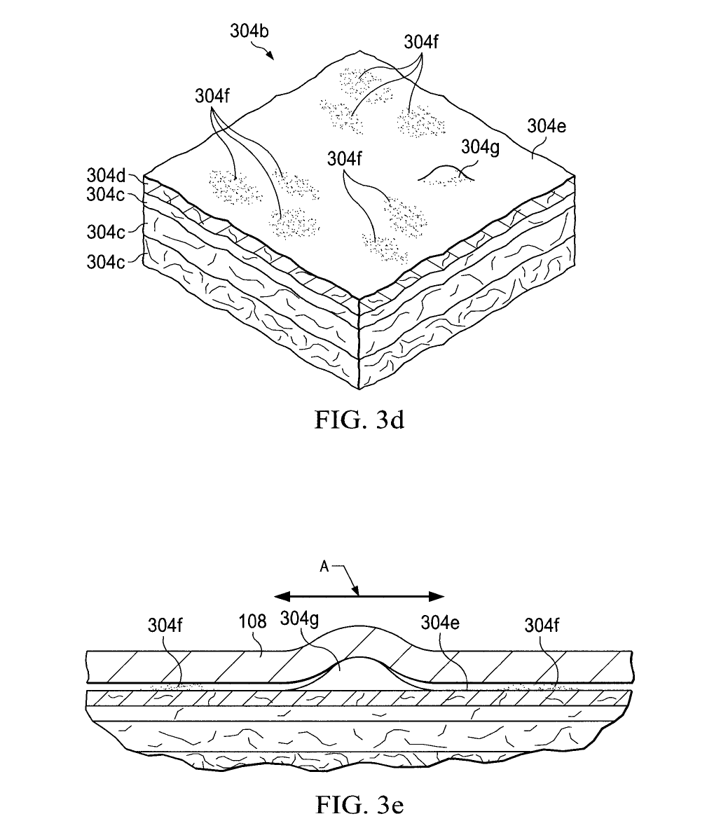

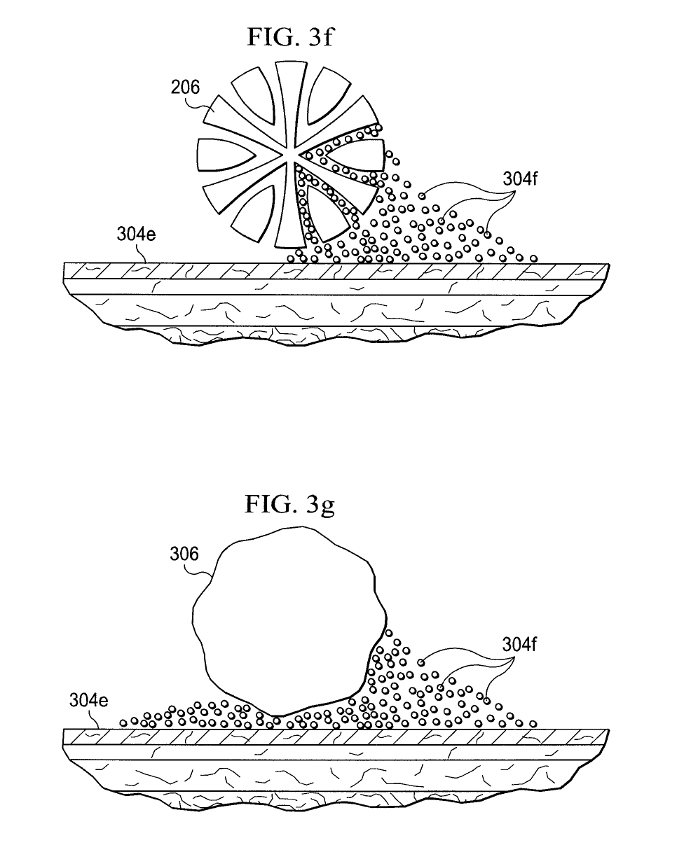

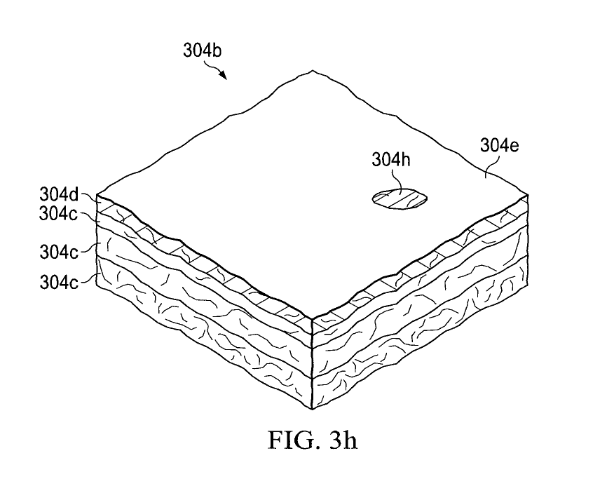

Referring now to FIGS. 3a and 3b, a method 300 for treating an affected skin area is illustrated. The method 300 begins at block 302 where a pillow with an exfoliating micro-fiber material is provided. In an embodiment, the pillow case 102, described above with reference to FIG. 1, is provided. A pillow 302a is positioned in the pillow housing 104 by moving the pillow 302a through the pillow entrance 106 until the pillow is located adjacent the inner surface 102a of the pillow case 102, as illustrated in FIG. 3b. As discussed above, block 302 of the method 300 may also be performed by replacing the pillow case 102 and pillow 302a with a pillow (e.g., the pillow 302a) that includes the exfoliation section 108 having the exfoliating micro-fiber material.

Referring now to FIGS. 3a, 3c, and 3d, the method 300 then proceeds to block 304 where skin is positioned in contact the exfoliating micro-fiber material. In the illustrated embodiment, a person 304a having one or more affected skin areas 304b, illustrated in FIG. 3c, may use the exfoliation/skin treatment system while they are sleeping or relaxing as is described below. The affected skin area 304b of the person 304a, illustrated in FIG. 3d, includes a plurality of adjacent lower epidermal layers 304c and an outer epidermal layer 304d. The outer epidermal layer 304d includes an outer surface 304e. The outer epidermal layer 304d and/or the outer surface 304e may include a skin disorder such as, for example, acne, pimples, and/or a variety of skin disorders known in the art. As illustrated, the outer epidermal layer 304d and/or outer surface 304e include a plurality of dead skin cells 304f and a pimple 304g. The person 304a may place the affected skin area 304b on the exfoliation section 108 of the pillow case 102, as illustrated in FIGS. 3c and 3e.

Referring now to FIGS. 3a, 3c, 3e, 3f, 3g, and 3h the method 300 then proceeds to block 306 where skin is abraded by moving the skin relative to the exfoliating micro-fiber material. In an embodiment, while the person 304a sleeps or relaxes, they may move the affected skin area 304b relative to the pillow 102 by, for example, rolling over (as illustrated in FIG. 3c) and/or moving their head around. Such movement results in the relative movement of the exfoliating section 108 of the pillow case 102 and the affected skin area 304b in, for example, the direction A illustrated in FIG. 3e. Relative movement of the exfoliating section 108 of the pillow case 102 and the affected skin area 304b will cause the micro-fibers in the exfoliating micro-fiber material to abrade the affected skin area 304b (e.g., through contact between the outer surface 304a of the affected skin area 304b and the edges 206b of the micro-fiber elements 206a on each micro-fiber 200, 202, and/or 204) to remove dead skin cells 304f from the outer surface 304e of the affected skin area 304b. Furthermore, each micro-fiber 206 may provide an electrostatic attraction to dead skin cells 304f and/or any skin cells that have been removed from the affected skin area 304b such that the dead skin cells are not only removed but also captured by the micro-fiber 206, as illustrated in FIG. 3f. This may be contrasted with prior art pillows that include fibers 306, illustrated in FIG. 3g, that either leave dead skin cells 304f on the outer surface 304a of the affected skin area 304b or simply push dead skin cells 304f to different locations on the outer surface 304a of the affected skin area 304b. In an embodiment, the exfoliating micro-fiber material may be chosen (e.g., having 70% polyester material and 30% polyamide material) such that the abrading that occurs is relatively ‘gentle’ and only abrades portions of the affected skin area 304b. For example, as illustrated in FIG. 3h, the affected skin area 304b is illustrated subsequent to the relative movement of the exfoliating section 108 of the pillow case 102 and the affected skin area 304b and the resulting abrasion of portions the affected skin area 304b. In an embodiment, the plurality of dead skin cells 304f have been removed from the affected skin area 304b due to the abrasion and/or captured by the micro-fibers 206 in the exfoliating section 108 of the pillow case 102. In an embodiment, the pimple 304g has been ruptured and/or removed from the affected skin area 304b due to the abrasion, leaving a portion 304h of the affected skin area 304b. In experimental embodiments, the abrading of portions of the affected skin area 304b (such as the dead skin cells 304f, the pimple 304g, etc) has been found to promote healing of the affected akin area 304b (e.g., portion 304b) such that the affected skin area 304b heals quicker than when using conventional methods. For example, the removal of the dead skin cells 304f from the affected skin area 304b prevents those dead skin cells 304f from clogging pores, which can cause pimples. Furthermore, the removal of dead skin cells allows the pores on the face to stay open, and oil does not get trapped in the pore to cause a pimple, but rather is released on the outer surface 304a of the affected skin area 304b and absorbed (e.g., by the exfoliating section 108 and/or other sections of the pillow case 102.)

Thus, an exfoliation/skin treatment system and method are provided that gently abrade an affected area of skin on a person while that person is sleeping in order to promote healing of the affected area of skin. The exfoliation/skin treatment system and method of the present disclosure are taught against in the prior art, but the Applicants have found that the abrading and possible rupturing of affected areas or portions of affected areas of skin helps to promote the healing of those affected areas, and provides advantages in skin treatment over conventional methods.

Furthermore, while exfoliation/skin treatment system and method described above is directed to a pillow or pillow case, bed sheets, blankets, and/or a variety of bed materials, exfoliation/skin treatment systems within the scope of the present disclosure may include wearable articles. For example, gloves, socks, and/or other wearable articles may include the exfoliating section (e.g., exfoliating section 108 discussed above) on an inner surface (e.g., the entire inner surface, a portion of the inner surface, etc.) of the wearable article that is in contact with a persons skin when they are wearing the wearable article, while the outer surface of the wearable article may include conventional, non-exfoliating surfaces known in the art. Similarly as discussed above, use of such exfoliating/skin treatment systems are taught against in the prior art but have been found to provide similar advantages to those explained above.

Although illustrative embodiments have been shown and described, a wide range of modification, change and substitution is contemplated in the foregoing disclosure and in some instances, some features of the embodiments may be employed without a corresponding use of other features. Accordingly, it is appropriate that the appended claims be construed broadly and in a manner consistent with the scope of the embodiments disclosed herein.

What is claimed is:

1. An exfoliation system, comprising:
a pillow case that defines a pillow housing;
an exfoliation section located on the pillow case opposite the housing; and
an exfoliating micro-fiber material included in the exfoliation section, wherein the exfoliating micro-fiber material includes a plurality of micro-fibers that are operable, in response to contact with and relative motion of an affected skin area, to abrade the affected skin area.

2. The system of claim 1, wherein the exfoliation section includes an entire outer surface of the pillow case that is opposite the pillow housing.

3. The system of claim 1, wherein the exfoliating micro-fiber material includes the plurality of micro-fibers in a weaved orientation.

4. The system of claim 1, wherein the exfoliating micro-fiber material each includes the plurality of micro-fibers each in a looped orientation.

5. The system of claim 1, wherein each of the plurality of microfibers includes a bundle of ultra-micro fibers.

6. The system of claim 1, wherein the plurality of micro-fibers are operable, in response to contact with and relative motion of the affected skin area, to rupture a portion of the affected skin area in order to promote healing of the affected skin area.

7. The system of claim 1, wherein the plurality of micro-fibers are operable, in response to contact with and relative motion of the affected skin area, to abrade the affected skin area to remove a plurality of dead skin cells from the affected skin area.

8. The system of claim 1, wherein the exfoliating micro-fiber material includes 70% polyester material and 30% polyamide material.

9. A skin treatment system, comprising:
a pillow;
an exfoliation section coupled to the pillow; and
an exfoliating micro-fiber material included in the exfoliation section, wherein the exfoliating micro-fiber material includes a plurality of micro-fibers that are operable, in response to contact with and relative motion of an affected skin area, to abrade the affected skin area.

10. The system of claim 9, wherein the exfoliation section includes an entire outer surface of the pillow.

11. The system of claim 9, wherein the exfoliating micro-fiber material includes the plurality of micro-fibers in a weaved orientation.

12. The system of claim 9, wherein the exfoliating micro-fiber material includes the plurality of micro-fibers each in a looped orientation.

13. The system of claim 9, wherein each of the plurality of microfibers each includes a bundle of ultra-micro fibers.

14. The system of claim 9, wherein the plurality of micro-fibers are operable, in response to contact with and relative motion of the affected skin area, to rupture a portion of the affected skin area in order to promote healing of the affected skin area.

15. The system of claim 9, wherein the plurality of micro-fibers are operable, in response to contact with and relative motion of the affected skin area, to abrade the affected skin area to remove a plurality of dead skin cells from the affected skin area.

16. The system of claim 9, wherein the exfoliating micro-fiber material includes 70% polyester material and 30% polyamide material.

17. A method for treating an affected skin area, comprising:
providing a pillow including an exfoliating micro-fiber material coupled to the pillow;
positioning the affected skin area in contact with the exfoliating micro-fiber material; and
abrading the affected skin area by moving the affected skin area relative to the exfoliating micro-fiber material.

18. The method of claim 17, further comprising:
rupturing a portion of the affected skin area in order to promote healing of the affected skin area.

19. The method of claim 17, further comprising:
removing a plurality of dead skin cells from the affected skin area.

20. The method of claim 17, wherein the exfoliating micro-fiber material includes 70% polyester material and 30% polyamide material

21. The method of claim 17, wherein the abrading is performed on the affected skin area during the sleep of a person having the affected skin area.
